(19) United States
(12) Patent Application Publication
(10) Pub. No.: US 2025/0332384 A1
(43) Pub. Date: Oct. 30, 2025

(54) FIXING DEVICE

(71) Applicant: HI-LEX CORPORATION, Hyogo (JP)

(72) Inventor: Shinsaku KOARASHI, Hyogo (JP)

(21) Appl. No.: 19/185,393

(22) Filed: Apr. 22, 2025

(30) Foreign Application Priority Data
Apr. 25, 2024 (JP) 2024-071835

(51) Int. Cl. A61M 25/02

(52) U.S. Cl. CPC A61M 25/02; A61M 2025/024; A61M 2025/028

(57) ABSTRACT

The fixing device of the present invention comprises: a main body comprising a tubular portion having an internal space; a sealing member composed of an elastic material, wherein the sealing member is arranged between an outer periphery of the insertion member IS and an inner surface of the tubular portion; and a locking member to be locked to the tubular portion in a state in which the sealing member is compressed in an axial direction; wherein the sealing member is configured to be in a tubular shape having an insertion portion for insertion member, and the fixing device further comprises: at least one rigid portion arranged between an outer periphery of the sealing member and an inner surface of the insertion portion for insertion member.

### CROSS-REFERENCE TO RELATED APPLICATIONS

This application claims priority to JP Application No. 2024-71835, filed Apr. 25, 2024, the disclosure of which is expressly incorporated by reference herein in its entirety.

### TECHNICAL FIELD

The present invention relates to a fixing device.

### BACKGROUND OF THE INVENTION

When an insertion member such as a medical tube and a driveline is led from inside of a living body to outside of the living body, for example, a fixing device as shown in JP 2020-81537 A is disclosed.

In the fixing device of JP 2020-81537 A, the insertion member is inserted into a main body portion to be mounted to the skin and is held liquid tightly by a plurality of members. Specifically, the fixing device comprises a main body portion, a pair of half-split contact members that sandwich an outer periphery of the insertion member, a nut-shaped movement regulating portion that is fastened to the main body portion, an inner sealing portion that is arranged between the movement regulating portion and the insertion member, and an outer sealing portion that is arranged between the movement regulating portion and the main body portion.

### SUMMARY OF THE INVENTION

The above-described fixing device can hold liquid tightly the insertion member, but there has been a demand for a simpler structure that can hold liquid tightly the insertion member.

Therefore, it is an object of the present invention to provide a fixing device that can hold liquid tightly an insertion member with a simple structure.

The fixing device of the present invention is a fixing device for fixing an insertion member to be inserted into a wall-like tissue of a living body and being mounted to the living body, the fixing device comprising: a main body comprising a tubular portion having an internal space through which the insertion member is inserted; a sealing member composed of an elastic material, wherein the sealing member is arranged between an outer periphery of the insertion member and an inner surface of the tubular portion in the internal space of the tubular portion; and a locking member to be locked to the tubular portion in a state in which the sealing member arranged in the internal space is compressed in an axial direction of the insertion member; wherein the sealing member is configured to be in a tubular shape having an insertion portion for insertion member to pass the insertion member, the fixing device further comprising: at least one rigid portion arranged between an outer periphery of the sealing member and an inner surface of the insertion portion for insertion member.

Moreover, the fixing device of the present invention is a fixing device for fixing an insertion member to be inserted into a wall-like tissue of a living body and being mounted to the living body, the fixing device comprising: a main body comprising a tubular portion having an internal space through which the insertion member is inserted; a sealing member composed of an elastic material, wherein the sealing member is arranged between an outer periphery of the insertion member and an inner surface of the tubular portion in the internal space of the tubular portion; and a locking member to be locked to the tubular portion in a state in which the sealing member arranged in the internal space is compressed in an axial direction of the insertion member; wherein the sealing member is configured in a tubular shape having an insertion portion for insertion member to pass the insertion member and the sealing member has a thick portion having a longer distance in a direction perpendicular to the axial direction and a thin portion having a shorter distance in the direction perpendicular to the axial direction than the thick portion.

According to the fixing device of the present invention, the insertion member can be held liquid tightly with a simple structure.

### BRIEF DESCRIPTION OF THE DRAWINGS

FIG. 1 is a side view of a fixing device according to one embodiment of the present invention.

FIG. 2 is a perspective view of the fixing device in FIG. 1.

FIG. 3 is an exploded perspective view of the fixing device in FIG. 1.

FIG. 4 is a view of the fixing device in FIG. 1, as viewed in an axial direction.

FIG. 5 is a cross-sectional view taken along the line V-V in FIG. 4.

FIG. 6 is a view of a main body used in the fixing device in FIG. 1, as viewed in an axial direction.

FIG. 7 is a perspective view of the main body in FIG. 6.

FIG. 8 is a cross-sectional view taken along the line VIII-VIII in FIG. 6.

FIG. 9 is a perspective view of a sealing member used in the fixing device in FIG. 1.

FIG. 10 is a partial enlarged view of a tubular portion with a sealing member mounted thereto, as viewed in an axial direction.

FIG. 11 is a schematic view showing a variation of the sealing member.

FIG. 12 is a schematic view showing another variation of the sealing member.

FIG. 13 is a partial enlarged view of a tubular portion with a sealing member mounted thereto, as viewed in an axial direction, in a variation of the fixing device of the present invention.

### DETAILED DESCRIPTION

The fixing device of one embodiment of the present invention will be described below with reference to the drawings. It should be noted that embodiments shown below are merely examples, and the fixing device of the present invention is not limited to the following embodiments.

It should be noted that, in the present specification, “perpendicular to A” and similar expressions do not only refer to a direction strictly perpendicular to A, but also refer to the direction including being substantially perpendicular to A. Moreover, in the present specification, “parallel to B” and similar expressions do not only refer to a direction strictly parallel to B, but also refer to the direction including being substantially parallel to B. In addition, in the present specification, “C-shape” and similar expressions do not only refer to a strict C-shape, but also refer to the shape including a shape visually associated with a C-shape (substantially a C-shape).

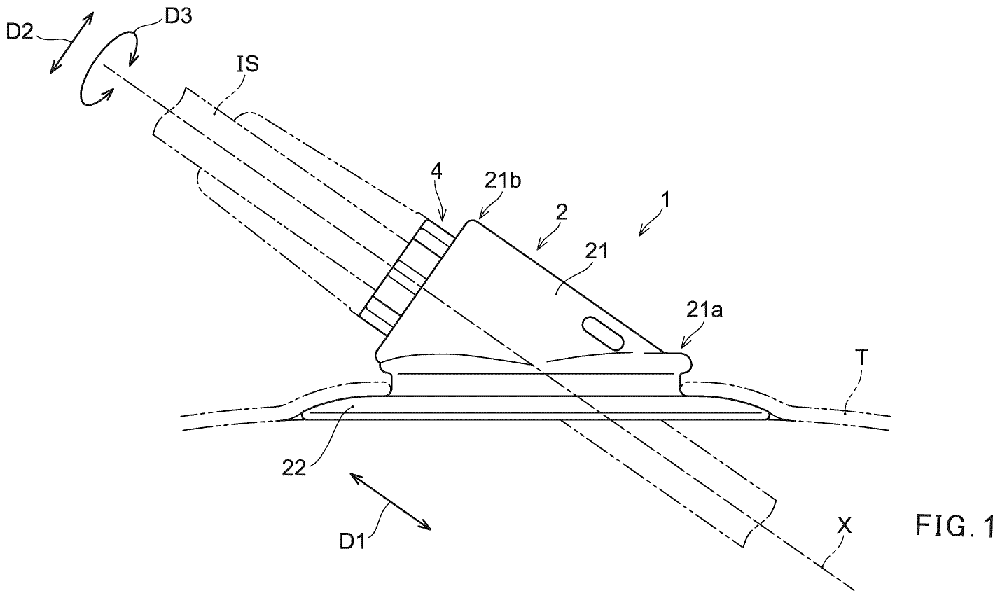

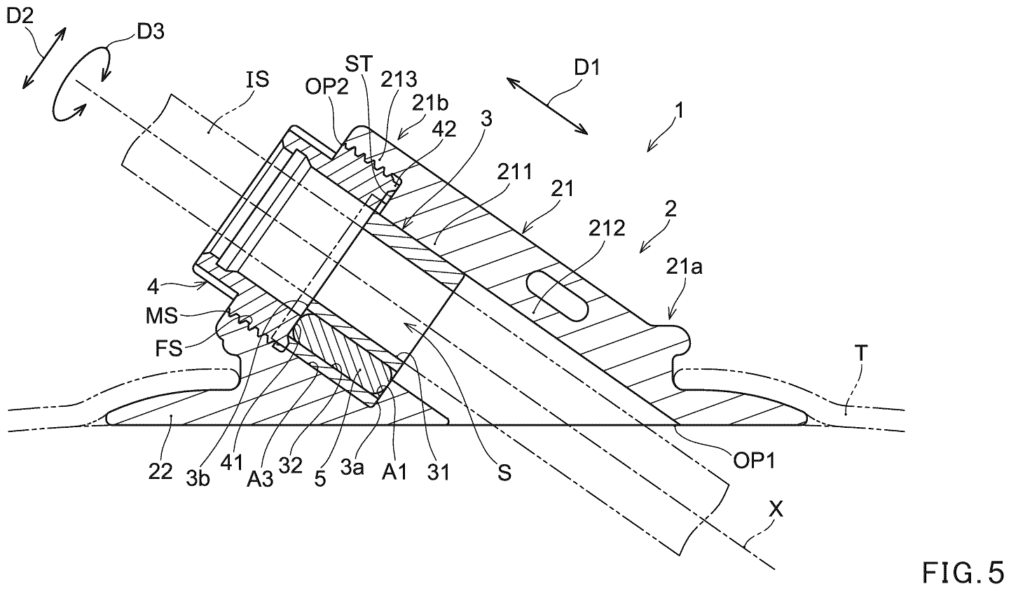

As shown in FIGS. 1 and 5, the fixing device 1 of the present embodiment fixes an insertion member IS to be inserted into a wall-like tissue T of a living body, and is mounted to the living body. In the present embodiment, as shown in FIGS. 1 and 5, the fixing device 1 is fixed to the wall-like tissue T of the living body by being embedded at a predetermined position into a predetermined depth in the wall-like tissue T in a state in which the insertion member IS is inserted through the fixing device 1.

In the present specification, the term “living body” refers to a body of a human or a non-human animal. The term “wall-like tissue” refers to any tissue such as a wall and a membrane of a living body having a predetermined thickness, into which an insertion member IS is inserted. Specifically, the wall-like tissue refers to such as skins (epidermis, dermis, subcutaneous tissue), a muscle layer, and various tissues inside of the living body that constitute various organs, as well as layers formed by combining them. In this embodiment, the wall-like tissue T into which the insertion member IS is inserted is a tissue including skins (epidermis, dermis, subcutaneous tissue) and a muscle layer. A fixing position (embedding position) in the wall-like tissue T to which the fixing device 1 is fixed can be changed as appropriate depending on a body type, etc. of a patient (living body) to which the fixing device 1 is fixed.

Applications of the fixing device 1 are not particularly limited as long as the fixing device 1 is used for fixing an insertion member IS to be inserted into a wall-like tissue T of a living body and being mounted to the living body. For example, the fixing device 1 can be used for a skin button (cuff member), etc. into which the insertion member IS is inserted. In this embodiment, the fixing device 1 is used when inserting into an abdominal wall-like tissue T a drive line (insertion member IS) used in a medical instrument (e.g., an artificial organ such as a ventricular assist device (VAD) and an artificial lung) implanted into a human body and leading the drive line from inside of the living body to outside of the living body of the human body. The drive line led out to outside of the living body is fixed at a predetermined position of the abdominal wall-like tissue T via the fixing device 1 as shown in FIGS. 1 and 5.

The insertion member IS is a linear member having a predetermined length that is inserted into a wall-like tissue T of a living body. It should be noted that the term “linear” for an insertion member IS means that the insertion member IS extends a predetermined length, regardless of whether the insertion member IS is hollow or solid. In this embodiment, the insertion member IS is a long member for medical use that is placed in a state where it penetrates through the wall-like tissue T of the living body. More specifically, the insertion member IS is a drive line for an artificial organ (ventricular assist device). One end of the insertion member IS is coupled to an artificial organ (not shown) placed at a predetermined position inside of the living body, and the other end is coupled to an equipment (such as a power source) placed outside of the living body.

An internal structure of the insertion member IS is not particularly limited. For example, the insertion member IS has a cooling water circulating passage for circulating a cooling water between an artificial organ inside of the living body and a pressure pump outside of the living body and a power cable for coupling the artificial organ inside of the living body to a power source outside of the living body. It should be noted that the insertion member IS may be configured to have only a power cable or have only a cooling water circulating passage. Moreover, the insertion member IS may be configured to have a member having functions other than those described above or may be configured as a hollow member (tube) having no members inside. When the insertion member IS is used as a hollow member that communicates between inside of the living body and outside of the living body, it may be used to deliver a drug for treatment on a treatment site from outside of the living body to inside of the living body.

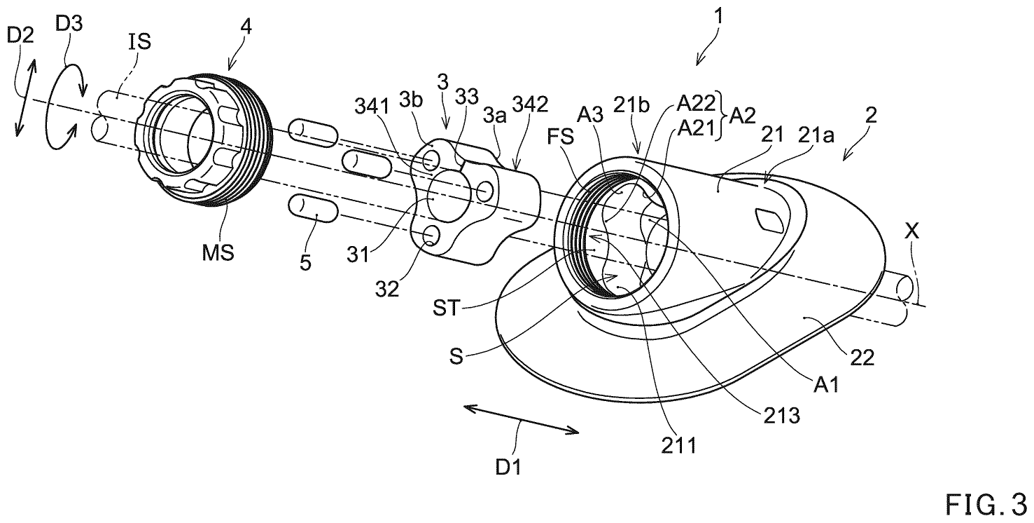

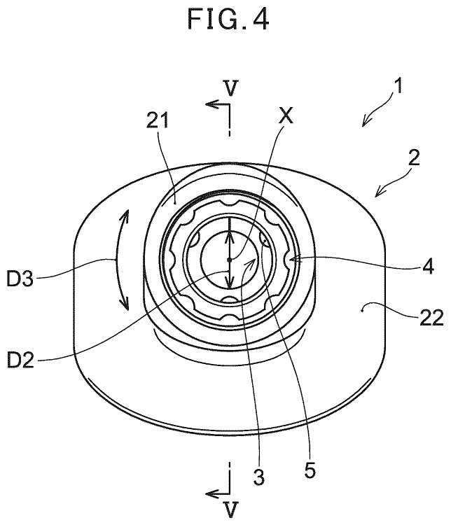

As shown in FIGS. 1 to 5, the fixing device 1 comprises a main body 2 comprising a tubular portion 21 having an internal space S (see FIG. 3) through which the insertion member IS is inserted, a sealing member 3 composed of an elastic material, wherein the sealing member 3 is arranged between an outer periphery of the insertion member IS and an inner surface of the tubular portion 21 in the internal space S of the tubular portion 21, and a locking member 4 to be locked to the tubular portion 21 in a state in which the sealing member 3 arranged in the internal space S is compressed in an axial direction D1 (see FIG. 1) of the insertion member IS.

For components of the fixing device 1 (e.g., the tubular portion 21, the sealing member 3, the locking member 4, a rigid portion 5 that will be mentioned later, etc.), a direction in which the insertion member IS extends in a state in which the insertion member IS inserted into the fixing device 1 is referred to as an axial direction D1 of the insertion member IS. Moreover, in a state in which the insertion member IS is inserted into the fixing device 1, an axial line passing through the center of the insertion member IS is referred to as an axis X (see FIG. 1). Furthermore, for the components of the fixing device 1, a direction perpendicular to the axial direction D1 is referred to as a direction away from the axis X or a radial direction D2 (see FIG. 1). It should be noted that, in the drawings, only one radial direction D2 is shown for convenience of illustration, but a radial direction is not limited to the direction shown in the drawings and may be any direction radiated from the axis X (a radial direction or a diameter direction of a circle centered on the axis X). Moreover, for the components of the fixing device 1, a direction rotating about the axis X (a direction around the axis X) is referred to as a circumferential direction D3 (see FIG. 1).

The main body 2 is a base portion of the fixing device 1, into which the insertion member IS is inserted internally. In the present embodiment, as shown in FIGS. 1 to 8, the main body 2 comprises a tubular portion 21 having an inner space S through which an insertion member IS is inserted and a fixing portion 22 to be mounted to a living body. In the present embodiment, the tubular portion 21 and the fixing portion 22 are formed integrally, but the tubular portion and the fixing portion may be formed separately from each other.

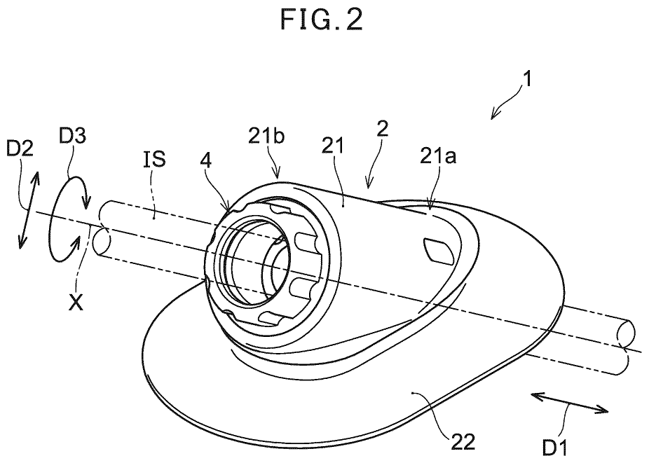

The fixing portion 22 is a portion to be fixed to a wall-like tissue T of a living body. Specifically, the fixing portion 22 is fixed around a hole (not shown) formed in the wall-like tissue T for inserting the insertion member IS (see FIGS. 1 and 5). A shape of the fixing portion 22 is not particularly limited as long as the fixing portion 22 can fix the fixing device 1 to the wall-like tissue T of the living body. In the present embodiment, as shown in FIGS. 2 to 4, 6, and 7, the fixing portion 22 is formed in a flange shape so as to surround the end of the tubular portion 21 of the main body 2. The flange-shaped fixing portion 22 is fixed to a skin by being embedded under the skin (wall-like tissue T) (see FIGS. 1 and 5).

A material of the fixing portion 22 is not particularly limited as long as the fixing portion 22 can fix the fixing device 1 to a wall-like tissue T of a living body. The fixing portion 22 is composed of, for example, a biocompatible material. As the material constituting the fixing portion 22, for example, a metal material having biocompatibility and a high corrosion resistance can be used, such as titanium and a titanium alloy. The surface of the fixing portion 22 may be subjected to surface treatment so as to improve adhesion to the wall-like tissue T. Moreover, as a variation, the fixing portion (for example, when the tubular portion and the fixing portion are separate bodies) may be composed of a sheet body composed of a non-absorbable material having biocompatibility. In this case, the non-absorbable material constituting the fixing portion can be a similar material as that used inside a body or at a wound site as a medical sheet. When the fixing portion is the above-described sheet body, the fixing portion is preferably composed of a flexible material. In this case, the fixing portion can follow movement of the wall-like tissue T such as the skin. When the fixing portion is composed of a flexible material, the material constituting the fixing portion can be, for example, at least one selected from the group consisting of a polyurethane resin, a polyamide resin, a polylactic acid resin, a polyolefin resin, a polyester resin, a fluororesin, a urea resin, a phenolic resin, an epoxy resin, a polyimide resin, an acrylic resin, a methacrylic resin, and derivatives thereof, and it can be preferably an expanded polytetrafluoroethylene (ePTFE).

The tubular portion 21 is a portion of the main body 2 through which the insertion member IS is inserted. As shown in FIGS. 3, 5 to 8, the tubular portion 21 has an internal space S through which the insertion member IS is inserted. In the present embodiment, the tubular portion 21 is configured to support the insertion member IS such as a drive line via the sealing member 3 with the insertion member IS inserted therethrough (see FIG. 5). The tubular portion 21 has a predetermined rigidity capable of supporting the insertion member IS with being inserted therethrough, and is composed of a biocompatible material. A material constituting the tubular portion 21 is not particularly limited, but it is preferably, for example, a metal material having biocompatibility and a high corrosion resistance, such as titanium and a titanium alloy, or a resin material having biocompatibility and a highly toughness, such as polyether ether ketone (PEEK). An overall shape of the tubular portion 21 is not particularly limited as long as the insertion member IS can be inserted therethrough. In the present embodiment, the tubular portion 21 is formed in a substantially cylindrical shape. Specifically, the tubular portion 21 extends inclining in a direction non-perpendicular to an extending direction of a flange-shaped fixing portion 22. It should be noted that the tubular portion 21 can be appropriately set depending on a type of an insertion member IS to be inserted, a site of a wall-like tissue T of a living body to which the fixing device 1 is mounted, or the like. The tubular portion 21 may extend vertically from the fixing portion 22.

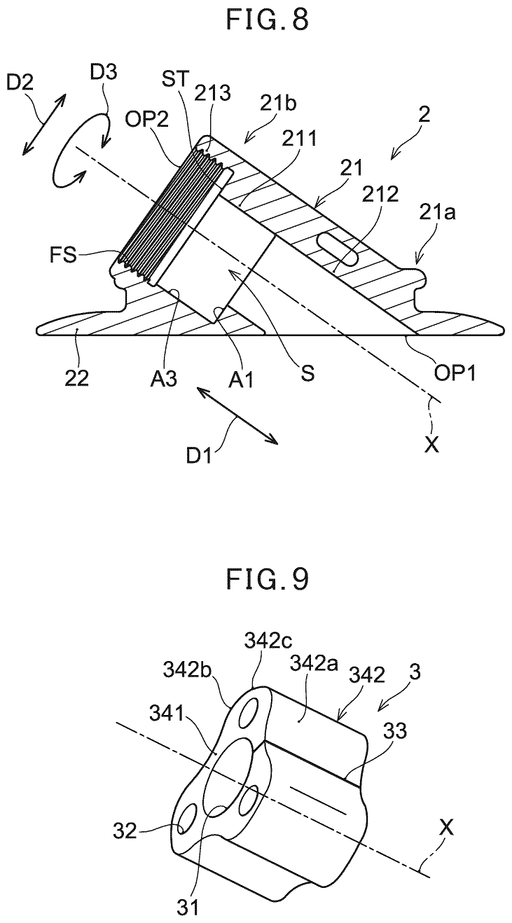

As shown in FIGS. 5 and 8, the tubular portion 21 has a first end 21a that is an end region at a side of the inside of a living body and a second end 21b that is an end region at a side of the outside of the living body. As shown in FIGS. 5 and 8, the tubular portion 21 has a first opening OP1 provided on the first end 21a side (at the side of the inside of the living body) and a second opening OP2 provided on the second end 21b side (at the side of the outside of the living body). In the present embodiment, an internal space S is formed between the first opening OP1 and the second opening OP2 (see FIG. 8). It should be noted that the internal space S is the entire space between the first opening OP1 and the second opening OP2, and includes spaces other than a space through which the insertion member IS passes (for example, a space in which the sealing member 3 is accommodated, a space around the insertion member IS, etc.). In the present embodiment, the insertion member IS is led out from the first opening OP1 of the first end 21a toward the inside of the living body. Moreover, the insertion member IS is led out from the second opening OP2 of the second end 21b toward the outside of the living body. A locking member 4 is mounted to the second end 21b, as shown in FIGS. 1, 2, and 5.

In the present embodiment, the tubular portion 21 comprises an accommodating portion 211 for accommodating the sealing member 3, as shown in FIGS. 5 to 8. Moreover, as shown in FIG. 5, the tubular portion 21 has an insertion portion 212 into which the insertion member IS is inserted, with a gap opened in a radial direction between an outer periphery of the insertion member IS and the insertion portion 212, on one side in an axial direction D1 of the tubular portion 21 with respect to the accommodating portion 211 (the living body side in a state in which the fixing device 1 is mounted to the living body). Furthermore, as shown in FIGS. 5 and 8, the tubular portion 21 has a locking member mounting portion 213 on the other side in the axial direction D1 of the tubular portion 21 with respect to the accommodating portion 211 (the side away from the living body in a state in which the fixing device 1 is mounted to the living body).

The accommodating portion 211 is a part of the tubular portion 21 in which the sealing member 3 is accommodated and arranged (see FIG. 5). When the sealing member 3 is compressed in the axial direction D1 as mentioned below in a state in which the sealing member 3 accommodated in the accommodating portion 211, the accommodating portion 211 comes into tight contact with the outer surface of the sealing member 3. This suppresses a bodily fluid such as blood from passing through the region of the sealing member 3 in the axial direction D1. A length of the accommodating portion 211 in the axial direction D1 is not particularly limited as long as the accommodating portion 211 can accommodate the sealing member 3 in a state in which the sealing member 3 can be compressed in the axial direction D1. The accommodating portion 211 may be configured to accommodate the entire sealing member 3 in the axial direction D1 or may be configured to accommodate a portion of the sealing member 3. In the present embodiment, as shown by the two-dot chain lines in FIG. 5, the length of the accommodating portion 211 in the axial direction D1 is shorter than a length of the sealing member 3 in the axial direction D1 before compression so that the other end of the sealing member 3 (the end on the locking member 4 side) protrudes from the accommodating portion 211 in the axial direction D1.

The inner surface of the accommodating portion 211 defines a space in which the sealing member 3 is accommodated. A shape of the inner surface of the accommodating portion 211 is not particularly limited as long as the inner surface of the accommodating portion 211 can come into tight contact with the outer surface of the sealing member 3 and can suppress a bodily fluid such as blood from passing through a position of the sealing member 3 in the axial direction D1. In the present embodiment, the inner surface of the accommodating portion 211 and the outer surface of the sealing member 3 have corresponding shapes to each other. The “corresponding shapes” mean that the inner surface shape of the accommodating portion 211 and the outer surface shape of the sealing member 3 are identical or similar to an extent that they can come into tight contact to each other in a state in which the sealing member 3 compressed and deformed in the axial direction D1, as mentioned below. Thus, before the sealing member 3 is compressed and deformed in the axial direction D1, the shape of the inner surface of the accommodating portion 211 and the shape of the outer surface of the sealing member 3 may be slightly different.

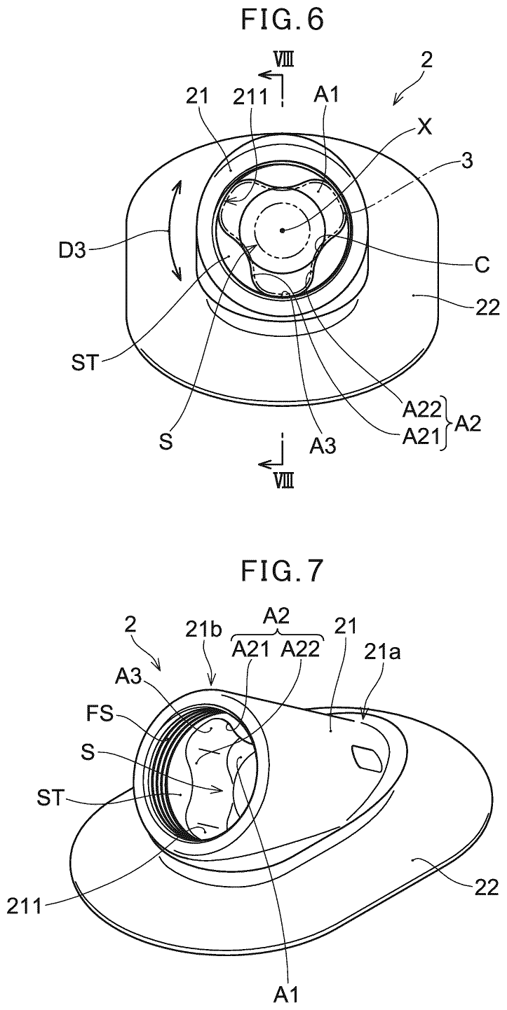

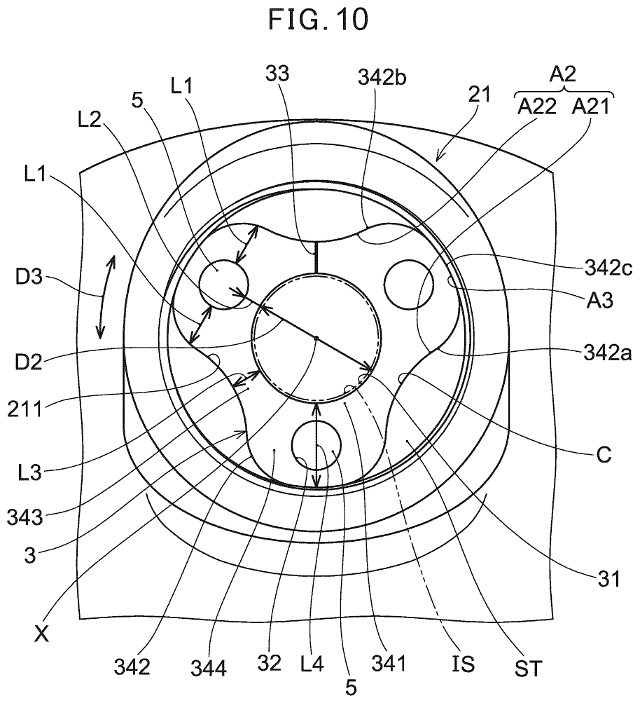

In the present embodiment, as shown in FIGS. 5 to 8, the accommodating portion 211 has an axial abutting portion A1 onto which one end face 3a (opposite to the locking member 4) of the sealing member 3 abuts in the axial direction D1 (see FIG. 5) when the sealing member 3 is accommodated in the accommodating portion 211. The axial abutting portion A1 positions the sealing member 3 by abutting onto one end face 3a of the sealing member 3 in the axial direction D1. Moreover, the axial abutting portion A1 also functions as a supporting portion that supports the end face 3a of the sealing member 3 when the sealing member 3 is compressed in the axial direction D1, as mentioned below. A shape and a structure of the axial abutting portion A1 are not particularly limited as long as it is configured to abut onto at least a portion of the end face 3a of the sealing member 3 in the axial direction D1. In the present embodiment, the axial abutting portion A1 is configured by an abutting surface extending perpendicularly to the axial direction D1. More specifically, the axial abutting portion A1 is configured by a flat surface abutting onto a portion of the end face 3a of the sealing member 3 which is on the outer side in the radial direction D2 (a part of a protruding portion 342 (see FIGS. 9 and 10) mentioned below) (in FIG. 6, the outline of the sealing member 3 is shown by the two-dot chain lines).

As shown in FIG. 10, the accommodating portion 211 comprises a circumferential abutting portion A2 that abuts onto the sealing member 3 in a circumferential direction of the insertion member IS, though the detail will be mentioned below. The circumferential abutting portion A2 is configured to face a portion of the sealing member 3 in the circumferential direction D3 and engage with the portion of the sealing member 3 in the circumferential direction D3. As a result, as mentioned below, even if a rotational force in the circumferential direction D3 is applied to the sealing member 3, the portion of the sealing member 3 engages with the circumferential abutting portion A2 in the circumferential direction D3, thereby restricting the sealing member 3 from rotating in the circumferential direction D3 with respect to the accommodating portion 211. In the present embodiment, the circumferential abutting portion A2 is configured to abut onto the protruding portion 342 of the sealing member 3, and the circumferential abutting portion A2 and the protruding portion 342 of the sealing member 3 engage with each other in the circumferential direction D3, thereby restricting the sealing member 3 from rotating in the circumferential direction with respect to the accommodating portion 211. In the present embodiment, as shown in FIGS. 6 and 10, the circumferential abutting portion A2 extends in a direction including a radial direction D2 component so as to engage with the protruding portion 342 of the sealing member 3 in the circumferential direction D3. A direction in which the circumferential abutting portion A2 extends (an angle with respect to the radial direction D2) is not particularly limited as long as it can abut onto and engage with the protruding portion 342 of the sealing member 3 in the circumferential direction D3. Moreover, in the present embodiment, the circumferential abutting portion A2 is constituted by a curved surface, but it may be constituted by a flat surface or a combination of a flat surface and a curved surface. In addition, in the present embodiment, as shown in FIGS. 6 and 10, the accommodating portion 211 has a first circumferential abutting portion A21 that engages with the protruding portion 342 of the sealing member 3 when a force in one rotational direction is applied to the sealing member 3 in the circumferential direction D3, and a second circumferential abutting portion A22 that engages with the protruding portion 342 of the sealing member 3 when a force in the other rotational direction is applied to the sealing member 3 in the circumferential direction D3. The first circumferential abutting portion A21 and the second circumferential abutting portion A22 are connected by a radial abutting portion A3. The radial abutting portion A3 extends along the circumferential direction D3, and engages with the outer surface of the sealing member 3 in the radial direction D2 when the sealing member 3 is compressed in the axial direction D1. The first circumferential abutting portion A21, the radial abutting portion A3, and the second circumferential abutting portion A22 define a concave portion in which one protruding portion 342 of the sealing member 3 is accommodated (see FIGS. 6 and 10). In the present embodiment, the accommodating portion 211 has a plurality of concave portions (three in the present embodiment) that accommodate a plurality of protruding portions 342 (three in the present embodiment) of the sealing member 3, respectively. One concave portion of the accommodating portion 211 and another concave portion adjacent to the one concave portion in the circumferential direction D3 are connected by a connecting portion C (see FIGS. 6 and 10). In the present embodiment, a plurality of concave portions, which accommodate the protruding portions 342 (thick portions 344) mentioned below, and a plurality of connecting portions C, which connect the plurality of concave portions and inwardly protrude, are constituted by curved surfaces. In this case, as mentioned below, when the sealing member 3 is compressed in the axial direction D1, the material of the sealing member 3 that is compressed and deformed is easy to move smoothly along the curved surface from the protruding portion 342 (thick portion 344) to the thin portion 343 (see FIG. 10). Thus, as mentioned below, a part of the thin portion 343 of the sealing member 3 makes it easier to seal the gap between the insertion member IS and the inner surface of the accommodating portion 211, thereby improving a sealing performance of the sealing member 3.

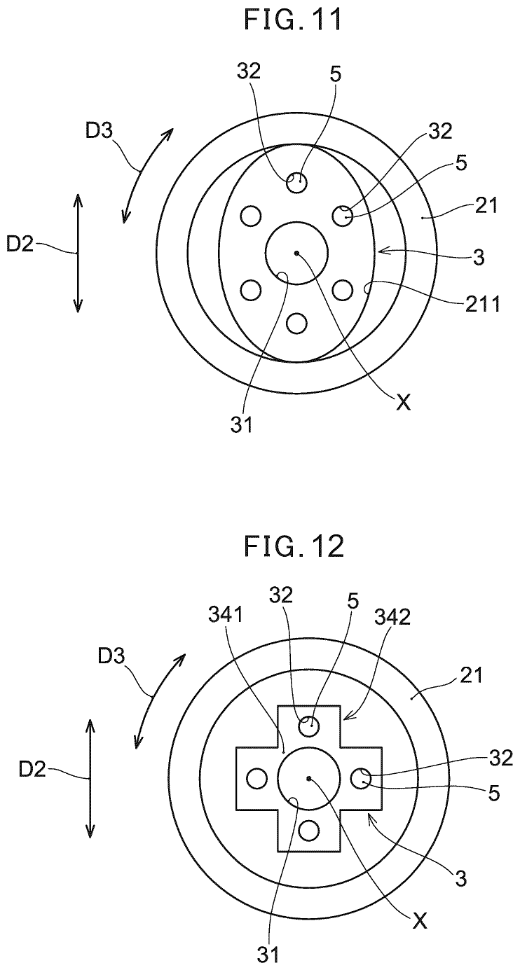

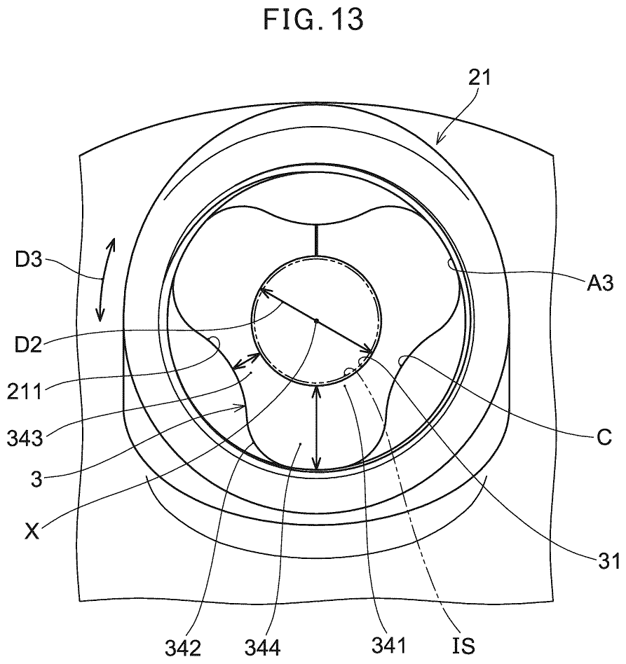

In the present embodiment, the accommodating portion 211 has a shape having a plurality of (three) concave portions to accommodate the plurality of (three) protruding portions 342 (thick portions 344) of the sealing member 3 (in other words, a shape in which a plurality of (three) protruding spaces extend outward in the radial direction D2 from a central space through which the insertion member IS passes). However, it should be noted that a shape of the accommodating portion is not limited to the shapes shown in FIGS. 6 and 10 as long as it can accommodate the sealing member 3. For example, in a case where the sealing member 3 has only one protruding portion (thick portion 344), the accommodating portion may have a shape having one concave portion. Thus, the space defined by the inner surface of the accommodating portion can be a shape in which one or more protruding spaces extend outward in the radial direction D2 from the central space through which the insertion member IS passes. More specifically, the space defined by the inner surface of the accommodating portion may have a keyhole shape in which one protruding space (concave portion) extends outward in the radial direction D2 from the central space through which the insertion member IS passes, or a cross shape (see FIG. 12), a gear shape, or a star shape in which a plurality of protruding spaces (concave portion s) extend. Moreover, in addition to the above-described shapes, the space defined by the inner surface of the accommodating portion may be a circle, an elliptical shape (see FIG. 11), a polygonal shape such as a triangle, a square, a pentagon, and a hexagon, or may be a combination of the shapes mentioned above and other shapes.

The locking member mounting portion 213 is a part of the tubular portion 21 to which the locking member 4 is mounted. In the present embodiment, the locking member mounting portion 213 engages with the locking member 4 in the axial direction D1 and suppresses the locking member 4 from being detached from the tubular portion 21. As a result, with the locking member 4 compressing the sealing member 3 in the axial direction D1, even if the locking member 4 receives a reaction force in the axial direction D1 (in a direction opposite to the compressing direction) from the sealing member 3, the locking member 4 is held at a predetermined position with respect to the tubular portion 21. A shape and a structure of the locking member mounting portion 213 are not particularly limited as long as the locking member mounting portion 213 can engage with the locking member 4 in the axial direction D1 and suppress the locking member 4 from being detached from the tubular portion 21. In the present embodiment, as shown in FIGS. 3 and 5, the locking member mounting portion 213 is constituted by an engaging concave portion having a female screw FS into which a locking member 4 having a male screw MS is screwed. Specifically, as shown in FIGS. 3, 5, 7, and 8, the locking member mounting portion 213 is formed in a tubular shape that is large enough to allow a portion of the locking member 4 to be inserted internally, and has a female screw FS on its inner surface. However, the locking member mounting portion can be modified as appropriate depending on a structure of the locking member, and may have a structure different from those shown in the drawings. For example, when the locking member is snap-fitted to the locking member mounting portion, the locking member mounting portion may be configured to have an engaged portion that engages with an engaging claw of the locking member in the axial direction D1, rather than the female screw.

In the present embodiment, as shown in FIGS. 5 to 8, the tubular portion 21 has a stopping portion ST that abuts onto a portion of the locking member 4 in the axial direction D1 when the locking member 4 is moved in the axial direction D1 with respect to the locking member mounting portion 213 and stops the locking member 4 at a predetermined position in the axial direction D1. With the stopping portion ST provided in the tubular portion 21, when the locking member 4 engages with the locking member mounting portion 213, a portion of the locking member 4 (one end of the locking member 4 in the axial direction D1 in the present embodiment) abuts onto the stopping portion ST, so that the locking member 4 stops at a predetermined axial position. An amount of compression of the sealing member 3 to be compressed by the locking member 4 can be thereby made constant, as mentioned below. Thus, the amount of compression of the sealing member 3 is suppressed from changing due to a degree of fastening by a user using the fixing device 1, etc. Therefore, deterioration of the sealing performance due to insufficient compression can be suppressed.

The locking member 4 is locked to the tubular portion 21 with the sealing member 3 arranged in the internal space S being compressed in the axial direction D1. In the present embodiment, when the locking member 4 is locked to the tubular portion 21, the locking member 4 moves relative to the tubular portion 21 in the axial direction D1. In the present embodiment, when the locking member 4 moves relative to the tubular portion 21, the locking member 4 is configured to press the sealing member 3 in the axial direction D1 and compress the sealing member 3 in the axial direction D1 (in FIG. 5, the position of the sealing member 3 before compression is indicated by the two-dot chain lines, and the sealing member 3 indicated by the solid lines in FIG. 5 is in a compressed state), though the detail will be mentioned below. The insertion member IS inserted through the sealing member 3 is held by the sealing member 3 due to deformation of the sealing member 3 when the sealing member 3 is compressed in the axial direction D1.

A shape and a structure of the locking member 4 are not particularly limited as long as the locking member 4 is configured to lock the sealing member 3 arranged in the internal space S to the tubular portion 21 in a state in which the sealing member 3 is compressed in the axial direction D1. In the present embodiment, as shown in FIGS. 3 and 5, the locking member 4 is constituted by a fastening member having a male screw MS on its outer periphery. However, as mentioned above, the locking member 4 may have another structure. For example, the locking member 4 may be configured to have an engaging portion such as an engaging claw to be snap-fitted to the locking member mounting portion 213 having an engaged portion.

As shown in FIG. 5, the locking member 4 has a pressing portion 41 configured to press an end face 3b of the sealing member 3 (the other end face in the axial direction D1, which is opposite to the one end face 3a in the axial direction D1). In the present embodiment, the pressing portion 41 is constituted by an end face of the locking member 4 that can come into contact with the end face 3b of the sealing member 3 (in FIG. 5, due to the cutting position of the cross section, it appears as if the pressing portion 41 and the end face 3b of the sealing member 3 are not in contact with each other, but indeed, a part of the pressing portion 41 is in contact with the end face 3b of the sealing member 3). A shape and a structure of the pressing portion are not particularly limited as long as the pressing portion can press the end face 3b of the sealing member 3. In the present embodiment, the pressing portion 41 is configured to be in surface contact with the end face 3b of the sealing member 3, but it may also be configured to be in localized contact with the end face 3b of the sealing member 3, such as point contact and line contact.

In the present embodiment, as shown in FIG. 5, the locking member 4 comprises a contact portion 42 that comes into contact with the stopping portion ST of the tubular portion 21 in the axial direction D1. In the present embodiment, the contact portion 42 is provided at one end of the locking member 4 in the axial direction D1. Specifically, as shown in FIG. 5, the contact portion 42 is constituted by a protruding portion (an annular protruding portion in the present embodiment) that protrudes in the axial direction D1 on the outer periphery of one end of the locking member 4 in the axial direction D1. In the present embodiment, the contact portion 42 is provided so as to protrude in the axial direction D1 with respect to the pressing portion 41, but the end face of the locking member 4 may be formed in a planar shape having no protruding portions, and both the pressing portion and the contact portion may be constituted by a flat end face of the locking member 4.

The sealing member 3 seals a gap generated between the outer surface of the insertion member IS and the inner surface of the tubular portion 21. Specifically, by arranging the sealing member 3 between the outer surface of the insertion member IS and the inner surface of the tubular portion 21, a bodily fluid such as blood is suppressed from moving from one side to the other in the axial direction D1 of the sealing member 3. The sealing member 3 is configured to be compressed in the axial direction D1 by the locking member 4. The sealing member 3 is compressed and deformed in the axial direction D1, so that the sealing member 3 comes into tight contact with the outer surface of the insertion member IS and the inner surface of the tubular portion 21. Therefore, it is suppressed that the bodily fluid such as blood moves from one side to the other in the axial direction D1 of the sealing member 3.

The sealing member 3 is composed of an elastic material that is elastically deformable. A material constituting the sealing member 3 is not particularly limited as long as the sealing member 3 can seal the gap generated between the outer surface of the insertion member IS and the inner surface of the tubular portion 21. For example, an elastic material having biocompatibility, such as silicone rubber, is appropriately used for the material of the sealing member 3.

As shown in FIGS. 3, 5, and 9, the sealing member 3 is configured to be in a tubular shape having an insertion portion 31 for insertion member to pass the insertion member IS. In the present embodiment, the insertion portion 31 for insertion member is a through hole that penetrates the sealing member 3 in the axial direction D1. The insertion portion 31 for insertion member is configured so that a cross section cut perpendicularly to the axial direction D1 is circular. When the insertion member IS is passed through the insertion portion 31 for insertion member, the insertion member IS is surrounded by the sealing member 3. In the present embodiment, as shown in FIGS. 3, 9, and 10, the sealing member 3 is provided with a cutout portion 33 that communicates from the outer surface of the sealing member 3 to the insertion portion 31 for insertion member so that the insertion member IS can be moved from the outer surface of the sealing member 3 in the radial direction D2 to the insertion portion 31 for insertion member. As a result, the sealing member 3 can be easily mounted to the insertion member IS by moving the insertion member IS in a direction perpendicular to the axial direction D1 (radial direction D2) with respect to the sealing member 3 (or by moving the sealing member 3 in the radial direction D2 with respect to the insertion member IS). It should be noted that the sealing member may be mounted to the insertion member IS from the end of the insertion member IS without providing any cutout portions in the sealing member.

In the present embodiment, a length in the axial direction D1 of the sealing member 3 is longer than a length in the axial direction D1 of the accommodating portion 211, and when the sealing member 3 is arranged in the accommodating portion 211, a portion of the sealing member 3 is configured to protrude from the accommodating portion 211 (see the two-dot chain lines in FIG. 5). In the present embodiment, a part of the tubular portion 21 between the axial abutting portion A1 and the stopping portion ST in the axial direction D1 is configured as an accommodating portion 211, and when the sealing member 3 is arranged in the accommodating portion 211, a portion of the sealing member 3 protrudes from the stopping portion ST to the other side in the axial direction D1 (protrudes beyond the stopping portion ST to the outside of the accommodating portion 211), as shown by the two-dot chain lines in FIG. 5. In the present embodiment, this protruding portion is pressed in the axial direction D1 by the locking member 4, so that the sealing member 3 is compressed in the axial direction D1. It should be noted that the sealing member 3 may not protrude from the accommodating portion 211. The sealing member 3 may be compressed in the axial direction D1 by the locking member 4 protruding toward the accommodating portion 211 side and entering into the accommodating portion 211.

In addition, in the present embodiment, as shown in FIGS. 5, 9, and 10, the sealing member 3 has an accommodating hole 32 for accommodating a rigid portion 5 mentioned below. The accommodating hole 32 extends along the axial direction DI in the sealing member 3. A shape and a structure of the accommodating hole 32 are not particularly limited as long as the accommodating hole 32 can accommodate the rigid portion 5 so that the rigid portion 5 can exhibit effects mentioned below. In the present embodiment, as shown in FIG. 5, the accommodating hole 32 is constituted by a through hole that penetrates the sealing member 3 in the axial direction D1. However, the accommodating hole may not be penetrated in the axial direction D1 (for example, it may be configured to be open on only one side or on only the other side in the axial direction D1) as long as the accommodating hole 32 can accommodate the rigid portion 5. A position at which the accommodating hole 32 is provided is not particularly limited. In the present embodiment, the accommodating hole 32 is provided in the thick portion 344 of the sealing member 3 so that a distance in the radial direction D2 from the accommodating hole 32 to the insertion portion for insertion member 31 and a distance in the radial direction D2 from the accommodating hole 32 to the outer surface of the sealing member 3 become substantially equal. It should be noted that when the rigid portion is not provided as in the variation shown in FIG. 13, no accommodating hole is provided.

An overall shape of the sealing member 3 is not particularly limited as long as the sealing member 3 can seal the gap generated between the outer surface of the insertion member IS and the inner surface of the tubular portion 21 and allows for arrangement of the rigid portion 5. In the present embodiment, as shown in FIGS. 9 and 10, the sealing member 3 comprises an inner peripheral portion 341 configured to surround the outer periphery of the insertion member IS and a protruding portion 342 that protrudes outward from the inner peripheral portion 341 in a direction perpendicular to the axial direction D1 of the sealing member 3 (radial direction). The inner peripheral portion 341 is a portion provided around the insertion portion 31 for insertion member with a predetermined thickness in the radial direction D2. The inner peripheral portion 341 surrounds the insertion member IS when the insertion member IS is inserted through the sealing member 3. Moreover, as shown in FIG. 10, the sealing member 3 has a thick portion 344 having a longer distance L4 in a direction perpendicular to the axial direction D1 (radial direction D2) and a thin portion 343 having a shorter distance L3 in the direction perpendicular to the axial direction D1 (radial direction D2) than the thick portion 344. The thick portion 344 is a portion of the sealing member 3 in the circumferential direction D3, where a thickness in the radial direction D2 is relatively thicker than the other portion (thin portion 343). The thin portion 343 is a portion of the sealing member 3 in the circumferential direction D3, where a thickness in the radial direction D2 is relatively thinner than the other portion (thick portion 344). In the present embodiment, the thin portion 343 is provided between the thick portions 344 in the circumferential direction D3. In the present embodiment, the thick portion 344 is a portion where the protruding portion 342 is provided, and strictly speaking, the thick portion 344 is a portion where the inner peripheral portion 341 and the protruding portion 342 are combined at the position where the protruding portion 342 is provided in the circumferential direction D3. In the present embodiment, the thin portion 343 is a portion of the inner peripheral portion 341 located between a pair of protruding portions 342 in the circumferential direction D3.

The protruding portion 342 is a portion that protrudes outward from the inner peripheral portion 341 in the radial direction D2. In the present embodiment, as shown in FIGS. 9 and 10, a plurality of (three) protruding portions 342 are provided to be spaced apart at predetermined intervals in the circumferential direction D3. The protruding portion 342 is configured to face and abut the circumferential abutting portion A2 of the accommodating portion 211 in the circumferential direction D3. As a result, when a force is applied to rotate the sealing member 3 in the circumferential direction D3 in the accommodating portion 211, the protruding portion 342 and the circumferential abutting portion A2 engage with each other, thereby restricting the rotation of the sealing member 3. Thus, when the fixing device 1 is in use, the insertion member IS arranged to come into tight contact with the inner side of the sealing member 3 is suppressed from being subjected to a twisting force around the axis X. In particular, when the locking member 4 is a fastening member, the sealing member 3 receives a force from the locking member 4 to rotate in the circumferential direction D3. In response to this force, the protruding portion 342 engages with the circumferential abutting portion A2 of the accommodating portion 211 in the circumferential direction D3, thereby suppressing the sealing member 3 from rotating around the axis X. Thus, twisting of the insertion member IS around the axis X is suppressed, and changes in position and angle of the insertion member IS extending into the body, which are caused by the twisting of the insertion member IS, are suppressed.

In the present embodiment, as shown in FIGS. 9 and 10, the protruding portion 342 comprises a first abutting portion 342a that abuts onto the first circumferential abutting portion A21 of the accommodating portion 211, a second abutting portion 342b that abuts onto the second circumferential abutting portion A22 of the accommodating portion 211, and a third abutting portion 342c that abuts onto the radial abutting portion A3 of the accommodating portion 211. The first abutting portion 342a and the second abutting portion 342b are connected in the circumferential direction D3 by the third abutting portion 342c. The first abutting portion 342a extends at an angle and with a size that allows it to be arranged along the first circumferential abutting portion A21 of the accommodating portion 211. The second abutting portion 342b extends at an angle and with a size that allows it to be arranged along the second circumferential abutting portion A22 of the accommodating portion 211. The third abutting portion 342c extends at an angle and with a size that allows it to be arranged along the radial abutting portion A3 of the accommodating portion 211.

It should be noted that the shape of the sealing member 3 corresponds to a shape of an accommodating portion, such as a shape of the accommodating portion of the present embodiment and a shape of the accommodating portion of the variation as mentioned above. Thus, the sealing member 3 can have a shape having one or more protruding portions that protrude outward in the radial direction D2 from the inner peripheral portion configured to surround the outer periphery of the insertion member IS. More specifically, the sealing member may have a keyhole shape having one protruding portion that protrudes outward from the inner peripheral portion in the radial direction D2, or a cross shape (see FIG. 12), a gear shape, or a star shape having a plurality of protruding portions. Moreover, in addition to the above-described shapes, the sealing member may be a circle, an elliptical shape (see FIG. 11), a polygonal shape such as a triangle, a square, a pentagon, and a hexagon, or may be a combination of the shapes mentioned above and other shapes.

In the present embodiment, the fixing device 1 further comprises at least one rigid portion 5 arranged between the outer periphery of the sealing member 3 and the inner surface of the insertion portion 31 for insertion member, as shown in FIGS. 3, 5, and 10. It should be noted that the rigid portion 5 is not an essential element, and the sealing member 3 may not be provided with a rigid portion as in the variation shown in FIG. 13. It should be noted that the variation shown in FIG. 13 has the same configuration as the embodiment shown in FIGS. 1 to 10 except that it does not have a rigid portion and an accommodating hole, and the explanations described with respect to FIGS. 1 to 10 can also be applied to the variation.

As shown in FIGS. 5 and 10, the rigid portion 5 is arranged at a predetermined position between the outer periphery of the sealing member 3 and the inner surface of the insertion portion 31 for insertion member in the sealing member 3. The rigid portion 5 may be molded integrally with the sealing member 3 or may be provided so as to be detachable from the sealing member 3. In the present embodiment, the rigid portion 5 is provided so as to be detachable from the sealing member 3 and is configured to be inserted into the accommodating hole 32 of the sealing member 3 at the time of use. In the present embodiment, the rigid portion 5 is accommodated in the accommodating hole 32 so as to be movable relative to the sealing member 3 in the axial direction D1. In this case, when the sealing member 3 is compressed and deformed in the axial direction D1, the rigid portion 5 does not move integrally with the sealing member 3, so that the deformation of the sealing member 3 is not restricted by the rigid portion 5, allowing the sealing member 3 to be deformed freely. Thus, the sealing member 3 easily comes into tight contact with the insertion member IS or the accommodating portion 211. Moreover, when the rigid portion 5 is not sufficiently pressed into the accommodating hole 32, the rigid portion 5 is pressed by the locking member 4 and can move to a predetermined position.

The rigid portion 5 is composed of a rigid material having a higher rigidity than the sealing member 3. It is preferable that the rigid portion 5 has a predetermined rigidity such that the rigid portion 5 is not substantially deformed by a pressure applied to the rigid portion 5 from the sealing member 3 when the sealing member 3 is compressed in the axial direction D1. A material constituting the rigid portion 5 is not particularly limited as long as it has a predetermined rigidity, but for example, a metal material having biocompatibility and a high corrosion resistance, such as titanium, a titanium alloy, and polyether ether ketone can be used.

In the present embodiment, as shown in FIGS. 5 and 10, a rigid portion 5 is provided between the outer periphery of the sealing member 3 and the inner surface of the insertion portion 31 for insertion member. As a result, when the sealing member 3 is compressed in the axial direction D1 by the locking member 4, since the rigid portion 5 having a high rigidity becomes a hard wall, a portion of the sealing member 3 between the rigid portion 5 and the inner surface of the insertion portion 31 for insertion member is less likely to deform toward the rigid portion 5, and deforms toward the insertion member IS inserted into the insertion portion 31 for insertion member. In other words, when the sealing member 3 is compressed in the axial direction D1 by the locking member 4, the rigid portion 5 functions as a core material that displaces a portion of the sealing member 3 toward the insertion member IS. Thus, if a gap is generated between the insertion portion 31 for insertion member and the outer surface of the insertion member IS, the sealing member 3 deforms preferentially toward the insertion member IS, closes the gap, and comes into tight contact with the insertion member IS. The insertion member IS can be thereby held liquid tightly with a simple configuration without complicating the configuration of the fixing device 1.

In addition, when the sealing member 3 is compressed in the axial direction D1 by the locking member 4, since the rigid portion 5 having a high rigidity becomes a hard wall, a portion of the sealing member 3 between the rigid portion 5 and the inner surface of the accommodating portion 211 is less likely to deform toward the rigid portion 5, and deforms so as to fill the gap between the inner surface of the accommodating portion 211 and the outer surface of the sealing member 3. In other words, when the sealing member 3 is compressed in the axial direction D1 by the locking member 4, the rigid portion 5 functions as a core material that displaces a portion of the sealing member 3 toward the inner surface of the accommodating portion 211. Thus, if a gap is generated between the outer surface of the sealing member 3 and the inner surface of the accommodating portion 211, the sealing member 3 deforms preferentially so as to fill the gap, closes the gap, and comes into tight contact with the inner surface of the accommodating portion 211. Thereby, the gap between the outer surface of the sealing member 3 and the inner surface of the accommodating portion 211 can be filled, and the sealing member 3 can be held liquid tightly in the accommodating portion 211, with a simple configuration without complicating the configuration of the fixing device 1.

In the present embodiment, as shown in FIG. 10, the sealing member 3 has a thin portion 343 and a thick portion 344. The thick portion 344 has a higher rigidity than the thin portion 343, and the thin portion 343 deforms more easily than the thick portion 344. As a result, the thick portion 344 becomes a hard wall, and the sealing member 3 is less likely to deform from the thin portion 343 toward the thick portion 344, and the thin portion 343 deforms so as to fill the gap between the sealing member 3 and the insertion member IS and the gap between the inner surface of the accommodating portion 211 and the outer surface of the sealing member 3. In other words, when the sealing member 3 is compressed in the axial direction D1 by the locking member 4, the thick portion 344 functions as a core material that displaces the thin portion 343 toward the insertion member IS and the inner surface of the accommodating portion 211. Thus, if a gap is generated between the sealing member 3 and the insertion member IS and between the sealing member 3 and the accommodating portion 211, the sealing member 3 deforms preferentially so as to fill the gap, closes the gap, and comes into tight contact with the insertion member IS and the inner surface of the accommodating portion 211. The sealing member 3 can be thereby held liquid tightly in the insertion member IS and the accommodating portion 211 with a simple configuration without complicating the configuration of the fixing device 1. It should be noted that this effect can be obtained not only by the aspects shown in FIGS. 1 to 12 in which the rigid portion 5 is provided, but also by the variation shown in FIG. 13 in which no rigid portion is provided.

In the present embodiment, a portion of the sealing member 3 provided between the rigid portion 5 and the insertion member IS (the portion where the dimension line L2 is inserted in FIG. 10) also functions as a thin portion. Thus, in addition to the thin portion 343 provided between the pair of protruding portions 342 in the circumferential direction D3, the portion that functions as a thin portion is also present in a portion of the protruding portions 342, so that the insertion member IS is pressed inward from more locations in the circumferential direction D3. Thus, a holding force and a sealing performance of the insertion member IS are further improved.

In the present embodiment, the thick portion 344 extends along the axial direction D1 of the sealing member 3 and is provided as a whole in the axial direction D1 of the sealing member 3. In this case, the thin portion 343 of the sealing member 3 easily comes into tight contact with the insertion member IS and the inner surface of the accommodating portion 211 as a whole in the axial direction D1. For example, if only a thin portion is provided as a whole in the axial direction D1 of the sealing member (if the sealing member is thin as a whole in the axial direction D1 and there is no thick portion), there is no portion (thick portion) that retains the shape of the thin portion and guides a direction of deformation. In this case, when the sealing member is compressed in the axial direction D1 by the locking member 4, only the end region of the sealing member that comes into contact with the locking member 4 is deformed. For this reason, in a configuration without a thick portion, for example, in FIG. 5, the central portion of the sealing member in the axial direction D1 and the end region opposite the locking member 4 are less likely to deform, and an adhesive force of the sealing member to the insertion member IS and the inner surface of the accommodating portion 211 becomes weakened. In the present embodiment, with the thick portion 344 provided as a whole in the axial direction D1 of the sealing member 3, the thin portion 343 of the sealing member 3 can come into tight contact with the insertion member IS and the inner surface of the accommodating portion 211 as a whole in the axial direction D1.

In the present embodiment, by using the rigid portion 5, when the sealing member 3 is compressed in the axial direction D1 with the locking member 4, the gaps between the insertion member IS and the sealing member 3 and between the sealing member 3 and the accommodating portion 211 are sealed liquid tightly. Therefore, before the sealing member 3 is compressed in the axial direction D1 by the locking member 4, i.e., when the sealing member 3 is moved in the axial direction D1 along the insertion member IS before the locking member 4 is mounted to the locking member mounting portion 213 or when the sealing member 3 is arranged in the accommodating portion 211 before the locking member 4 is mounted to the locking member mounting portion 213, the sealing member 3 can be easily moved in the axial direction D1. Thus, an assembly work of the fixing device 1 can be facilitated.

In the present embodiment, a total of three rigid portions 5 are provided, but the number of rigid portions 5 can be changed as appropriate depending on a shape and a structure of the sealing member 3. In the present embodiment, the fixing device 1 has a plurality of rigid portions 5 arranged to be spaced apart from each other in the circumferential direction D3 of the insertion member IS. In this case, with the plurality of rigid portions 5 arranged in the circumferential direction D3 of the insertion member IS, the sealing member 3 comes into tight contact around the insertion member IS in a well-balanced manner. In order to obtain a uniform liquid tightness in the circumferential direction D3, it is preferable that three or more rigid portions 5 are provided at equal intervals in the circumferential direction D3.

The shape of the rigid portion 5 can be changed as appropriate depending on a shape and a structure of the sealing member 3, such as the accommodating hole 32 of the sealing member 3. In the present embodiment, as shown in FIGS. 3 and 5, the rigid portion 5 is formed in a generally cylindrical shape extending in the axial direction D1, but may have other shapes, such as a polygonal column and a plate shape. In the present embodiment, both ends of the rigid portion 5 in the axial direction DI are formed to have a curved surface such as a hemispherical surface. In this case, the rigid portion 5 is easily pushed into the accommodating hole 32 of the sealing member 3 when inserted therein. The rigid portion 5 may be configured so that a cross section perpendicular to the axial direction D1 of the rigid portion 5 is larger than a cross section of the accommodating hole 32 so that the rigid portion 5 is press-fitted into the accommodating hole 32. In this case, when the rigid portion 5 is assembled to the sealing member 3, the rigid portion 5 is less likely to come off. Moreover, the cross section of the rigid portion 5 perpendicular to the axial direction D1 may be smaller than the cross section of the accommodating hole 32, and a clearance may be provided between the outer surface of the rigid portion 5 and the accommodating hole 32. In this case, the rigid portion 5 can be smoothly inserted into the accommodating hole 32 of the sealing member 3. A length of the rigid portion 5 in the axial direction D1 is preferably such that the rigid portion 5 is not compressed in the axial direction D1 when the sealing member 3 is compressed in the axial direction D1. In the present embodiment, the length of the rigid portion 5 in the axial direction D1 is preferably shorter than a length between the axial abutting portion A1 and the pressing portion 41 of the locking member 4 with the sealing member 3 compressed.

In the present embodiment, as shown in FIG. 3, three rigid portions 5 are provided independently of one another, but for example, three rigid portions may be connected to one another at the ends in the axial direction D1 and constituted by a single member. Furthermore, the rigid portion may be provided integrally with the main body 2. For example, the rigid portion may be provided so as to protrude from the axial abutting portion A1 of the main body 2 toward the other side in the axial direction D1.

In the present embodiment, as shown in FIG. 10, the sealing member 3 is configured so that a distance L1 in the circumferential direction D3 from the rigid portion 5 to the circumferential abutting portion A2 is longer than a distance L2 from the rigid portion 5 to the inner surface of the insertion portion 31 for insertion member of the sealing member 3. As the distance L1 in the circumferential direction D3 from the rigid portion 5 to the circumferential abutting portion A2 is longer (compared to the distance L2), rigidity of a portion from the rigid portion 5 to the circumferential abutting portion A2 is enhanced. Therefore, when a force is applied to the sealing member 3 to rotate around the axis X, the sealing member 3 becomes less likely to deform in the circumferential direction D3, making it easier to restrict the rotation of the sealing member 3. Furthermore, as the distance L2 (in the radial direction D2) from the rigid portion 5 to the inner surface of the insertion portion 31 for insertion member of the sealing member 3 is shorter (compared to the distance L1), rigidity of a portion from the rigid portion 5 to the inner surface of the insertion portion 31 for insertion member of the sealing member 3 is reduced, making the sealing member 3 to easily deform. The sealing member 3 thereby more easily comes into tight contact with the outer periphery of the insertion member IS, so that a holding performance and a sealing performance of the insertion member IS can be further improved. In the present embodiment, a distance in the radial direction D2 from the rigid portion 5 to the inner surface of the accommodating portion 211 is equivalent to the distance L2 in the radial direction D2 from the rigid portion 5 to the inner surface of the insertion portion 31 for insertion member of the sealing member 3 (0.8 to 1.2 times the distance L2, preferably equal thereto). In this case, as the distance in the radial direction D2 from the rigid portion 5 to the inner surface of the accommodating portion 211 is shorter (compared to the distance L1), rigidity of a portion from the rigid portion 5 to the inner surface of the accommodating portion 211 is reduced, making the sealing member 3 to easily deform. The sealing member 3 thereby more easily comes into tight contact with the inner surface of the accommodating portion 211, so that a sealing performance of the sealing member 3 can be further improved.

In the present embodiment, the locking member 4 is configured so that the inner diameter of the locking member 4 is larger than the inner diameter of the insertion portion 31 for insertion member (the outer diameter of the insertion member IS). This configuration is intended for inserting a larger diameter portion than a tubular portion of the insertion member IS, such as a connector pin provided at the end of the insertion member IS, such as the drive line, which is connected to an equipment not shown in the figures. In this manner, in the case where the inner diameter of the locking member 4 is larger than the inner diameter of the insertion portion 31 for insertion member (the outer diameter of the insertion member IS) and the locking member 4 does not come into contact with the entire end face 3b of the sealing member 3, when the insertion member IS is pulled in the axial direction D1 (for example, in the upper left direction in FIG. 5), a force is applied to the sealing member 3 to deform the sealing member 3 so as to move following the insertion member IS. In the present embodiment, the rigid portion 5 provided in the sealing member 3 comes into contact with the end face (pressing portion 41) of the locking member 4 against this force, thereby restricting deformation of the sealing member 3. Thus, even if the entire end face 3b of the sealing member 3 is not in contact with the locking member 4, the rigid portion 5 having a high rigidity can suppress the sealing member 3 from being significantly deformed and damaged, and from coming out of the accommodating portion 211, when the insertion member IS is pulled in the axial direction D1. It should be noted that the inner diameter of the locking member is not particularly limited as long as it allows for insertion of the insertion member IS, and may be the same size as the inner diameter of the insertion portion for insertion member.

Next, a method of using the fixing device 1 of the present embodiment (a method of fixing the fixing device 1) will be described using an example in which the insertion member is a drive line (hereinafter referred to as a drive line IS) and the fixing device 1 is mounted to the drive line IS led out through a hole provided in the skin that is a wall-like tissue (hereinafter referred to as the skin T) to an outside of a body. It should be noted that the following descriptions are merely examples, and the fixing device of the present invention is not limited to the following descriptions.

First, the drive line IS led out from the skin T to the outside of the body is inserted through the tubular portion 21 of the main body 2 (see FIG. 3). Further, the drive line IS is inserted through the through hole of the locking member 4. When the drive line IS is inserted through the tubular portion 21 of the main body 2, the main body 2 is arranged at a predetermined position on the skin T. Specifically, the fixing portion 22 of the main body 2 is embedded under the skin T (see FIGS. 1 and 5).

Next, as shown in FIG. 3, the sealing member 3 is mounted to the drive line IS. In the present embodiment, the sealing member 3 is pressed toward the drive line IS in a direction perpendicular to the axis X from a portion of the cutout portion 33 of the sealing member 3, so that the sealing member 3 can be connected to the drive line IS. Next, the sealing member 3 is accommodated in the accommodating portion 211 of the tubular portion 21. Accommodation of the sealing member 3 in the accommodating portion 211 is performed by moving the sealing member 3 in the axial direction D1 along the drive line IS until the end face 3a of the sealing member 3 abuts onto the axial abutting portion A1. The rigid portion 5 may be mounted to the sealing member 3 before the sealing member 3 is mounted to the drive line IS or may be mounted after the sealing member 3 is mounted to the drive line IS. Moreover, the rigid portion 5 may be mounted to the sealing member 3 before the sealing member 3 is accommodated in the accommodating portion 211 or may be mounted to the sealing member 3 after the sealing member 3 is accommodated in the accommodating portion 211. After the sealing member 3 is accommodated in the accommodating portion 211 and before the locking member 4 is mounted to the locking member mounting portion 213, the sealing member 3 slightly protrudes from the accommodating portion 211 to the locking member 4 side in the axial direction D1, as shown by the two-dot chain lines in FIG. 5. That is, the sealing member 3 is in a state of not being compressed in the axial direction D1, and there are slight clearances between the inner surface of the sealing member 3 and the outer surface of the drive line IS and between the outer surface of the sealing member 3 and the inner surface of the accommodating portion 211, or they come into contact with each other with a low adhesive force. Therefore, the sealing member 3 can be smoothly moved relative to the drive line IS in the axial direction DI and the sealing member 3 can be smoothly accommodated in the accommodating portion 211.

When the sealing member 3 is accommodated in the accommodating portion 211, the locking member 4 is moved in the axial direction D1 toward the locking member mounting portion 213 of the tubular portion 21. When the locking member 4 reaches the locking member mounting portion 213, the locking member 4 is screwed into the locking member mounting portion 213. As the locking member 4 is screwed into the locking member mounting portion 213, the locking member 4 moves in the axial direction D1 with respect to the locking member mounting portion 213, and the pressing portion 41 of the locking member 4 abuts onto the end face 3b of the sealing member 3. When the locking member 4 is further screwed into the locking member mounting portion 213 in this state, the sealing member 3 is compressed in the axial direction D1 by the pressing portion 41 of the locking member 4. When the sealing member 3 is compressed in the axial direction D1, since the rigid portion 5 having a high rigidity becomes a hard wall, the portion of the sealing member 3 between the rigid portion 5 and the inner surface of the insertion portion 31 for insertion member deforms toward the drive line IS. Moreover, since the rigid portion 5 having a high rigidity becomes a hard wall, the portion between the rigid portion 5 and the inner surface of the accommodating portion 211 deforms so as to fill the gap between the inner surface of the accommodating portion 211 and the outer surface of the sealing member 3. As a result, the sealing member 3 comes into tight contact with the drive line IS and the accommodating portion 211 to complete holding of the drive line IS and sealing of the fixing device 1.

When the locking member 4 is screwed into the locking member mounting portion 213 as mentioned above, the locking member 4 rotates around the axis X with being in contact with the end face 3b of the sealing member 3, so that the sealing member 3 receives a force to rotate around the axis X from the pressing portion 41 of the locking member 4. In response to this rotating force, the protruding portion 342 of the sealing member 3 abuts onto the circumferential abutting portion A21, thereby suppressing the rotation of the sealing member 3. Thus, the sealing member 3 and the drive line IS come into tight contact with each other by compression of the sealing member 3 in the axial direction D1, but the rotation of the sealing member 3 around the axis X is suppressed. This suppresses twisting of the drive line IS around the axis X along with the rotation of the sealing member 3, and suppresses changes in position and angle of the drive line IS extending inside the body which are caused by the twisting of the drive line IS.

Although the embodiments of the present invention have been described above, the present invention is not limited to the above-described embodiments. It should be noted that the above-described embodiments mainly explain the inventions having the following configurations.

(1) A fixing device for fixing an insertion member to be inserted into a wall-like tissue of a living body and being mounted to the living body, the fixing device comprising: a main body comprising a tubular portion having an internal space through which the insertion member is inserted;
a sealing member composed of an elastic material, wherein the sealing member is arranged between an outer periphery of the insertion member and an inner surface of the tubular portion in the internal space of the tubular portion; and
a locking member to be locked to the tubular portion in a state in which the sealing member arranged in the internal space is compressed in an axial direction of the insertion member;
wherein the sealing member is configured to be in a tubular shape having an insertion portion for insertion member to pass the insertion member, the fixing device further comprising:
at least one rigid portion arranged between an outer periphery of the sealing member and an inner surface of the insertion portion for insertion member.

(2) A fixing device for fixing an insertion member to be inserted into a wall-like tissue of a living body and being mounted to the living body, the fixing device comprising:
a main body comprising a tubular portion having an internal space through which the insertion member is inserted;
a sealing member composed of an elastic material, wherein the sealing member is arranged between an outer periphery of the insertion member and an inner surface of the tubular portion in the internal space of the tubular portion; and
a locking member to be locked to the tubular portion in a state in which the sealing member arranged in the internal space is compressed in an axial direction of the insertion member;
wherein the sealing member is configured in a tubular shape having an insertion portion for insertion member to pass the insertion member and the sealing member has a thick portion having a longer distance in a direction perpendicular to the axial direction and a thin portion having a shorter distance in the direction perpendicular to the axial direction than the thick portion.

(3) The fixing device of (1) or (2),
wherein the sealing member comprises an inner peripheral portion configured to surround the outer periphery of the insertion member and a protruding portion that protrudes outward from the inner peripheral portion in a direction perpendicular to the axial direction of the sealing member, wherein the tubular portion comprises an accommodating portion for accommodating the sealing member, and
wherein the accommodating portion comprises a circumferential abutting portion that abuts onto the protruding portion of the sealing member in a circumferential direction of the insertion member.

(4) The fixing device of any one of (1) to (3),
wherein the fixing device further comprises at least one rigid portion arranged in the thick portion.

(5) The fixing device of any one of (1) to (4),
wherein the fixing device comprises at least one rigid portion arranged between an outer periphery of the sealing member and an inner surface of the insertion portion for insertion member, and
wherein a distance L1 in the circumferential direction from the rigid portion to the circumferential abutting portion is longer than a distance L2 from the rigid portion to the inner surface of the insertion portion for insertion member of the sealing member.

(6) The fixing device of any one of (1) to (5),
wherein the fixing device has a plurality of rigid portions arranged to be spaced apart from each other in the circumferential direction of the insertion member.

(7) The fixing device of any one of (1) to (6),
wherein the sealing member has an accommodating hole for accommodating the rigid portion, and the rigid portion is accommodated in the accommodating hole so as to be movable relative to the sealing member in the axial direction.

1. A fixing device for fixing an insertion member to be inserted into a wall-like tissue of a living body and being mounted to the living body, the fixing device comprising:
a main body comprising a tubular portion having an internal space through which the insertion member is inserted;
a sealing member composed of an elastic material, wherein the sealing member is arranged between an outer periphery of the insertion member and an inner surface of the tubular portion in the internal space of the tubular portion; and
a locking member to be locked to the tubular portion in a state in which the sealing member arranged in the internal space is compressed in an axial direction of the insertion member;
wherein the sealing member is configured to be in a tubular shape having an insertion portion for insertion member to pass the insertion member, the fixing device further comprising:
at least one rigid portion arranged between an outer periphery of the sealing member and an inner surface of the insertion portion for insertion member.

2. A fixing device for fixing an insertion member to be inserted into a wall-like tissue of a living body and being mounted to the living body, the fixing device comprising:
a main body comprising a tubular portion having an internal space through which the insertion member is inserted;
a sealing member composed of an elastic material, wherein the sealing member is arranged between an outer periphery of the insertion member and an inner surface of the tubular portion in the internal space of the tubular portion; and
a locking member to be locked to the tubular portion in a state in which the sealing member arranged in the internal space is compressed in an axial direction of the insertion member;
wherein the sealing member is configured in a tubular shape having an insertion portion for insertion member to pass the insertion member and the sealing member has a thick portion having a longer distance in a direction perpendicular to the axial direction and a thin portion having a shorter distance in the direction perpendicular to the axial direction than the thick portion.

3. The fixing device of claim 1,
wherein the sealing member comprises an inner peripheral portion configured to surround the outer periphery of the insertion member and a protruding portion that protrudes outward from the inner peripheral portion in a direction perpendicular to the axial direction of the sealing member, wherein the tubular portion comprises an accommodating portion for accommodating the sealing member, and
wherein the accommodating portion comprises a circumferential abutting portion that abuts onto the protruding portion of the sealing member in a circumferential direction of the insertion member.

4. The fixing device of claim 2, wherein the fixing device further comprises at least one rigid portion arranged in the thick portion.

5. The fixing device of claim 3,
wherein the fixing device comprises at least one rigid portion arranged between an outer periphery of the sealing member and an inner surface of the insertion portion for insertion member, and
wherein a distance L1 in the circumferential direction from the rigid portion to the circumferential abutting portion is longer than a distance L2 from the rigid portion to the inner surface of the insertion portion for insertion member of the sealing member.

6. The fixing device of claim 1, wherein the fixing device has a plurality of rigid portions arranged to be spaced apart from each other in the circumferential direction of the insertion member.

7. The fixing device of claim 1, wherein the sealing member has an accommodating hole for accommodating the rigid portion, and the rigid portion is accommodated in the accommodating hole so as to be movable relative to the sealing member in the axial direction.

8. The fixing device of claim 2,
wherein the sealing member comprises an inner peripheral portion configured to surround the outer periphery of the insertion member and a protruding portion that protrudes outward from the inner peripheral portion in a direction perpendicular to the axial direction of the sealing member, wherein the tubular portion comprises an accommodating portion for accommodating the sealing member, and
wherein the accommodating portion comprises a circumferential abutting portion that abuts onto the protruding portion of the sealing member in a circumferential direction of the insertion member.

9. The fixing device of claim 8,
wherein the fixing device comprises at least one rigid portion arranged between an outer periphery of the sealing member and an inner surface of the insertion portion for insertion member, and
wherein a distance L1 in the circumferential direction from the rigid portion to the circumferential abutting portion is longer than a distance L2 from the rigid portion to the inner surface of the insertion portion for insertion member of the sealing member.

10. The fixing device of claim 4, wherein the fixing device has a plurality of rigid portions arranged to be spaced apart from each other in the circumferential direction of the insertion member.

11. The fixing device of claim 4, wherein the sealing member has an accommodating hole for accommodating the rigid portion, and the rigid portion is accommodated in the accommodating hole so as to be movable relative to the sealing member in the axial direction.
